(19) United States
(12) Patent Application Publication
(10) Pub. No.: US 2025/0025621 A1
(43) Pub. Date: Jan. 23, 2025

(54) Surgical Suction Tube and Method

(71) Applicant: Michael T. Purslow, McCall, ID (US)

(72) Inventor: Michael T. Purslow, McCall, ID (US)

(21) Appl. No.: 18/777,977

(22) Filed: Jul. 19, 2024

Related U.S. Application Data
(60) Provisional application No. 63/527,725, filed on Jul. 19, 2023.

(51) Int. Cl. A61M 1/00; A61B 90/30; A61M 25/00; A61M 25/01

(52) U.S. Cl. CPC A61M 1/87; A61M 25/007; A61M 25/0102; A61B 90/30; A61M 2205/587; A61M 2205/8206

(57) ABSTRACT

A surgical suction device is provided having an elongate unitary construction lumen body and a clearing apparatus. The lumen body has a handle and a suction tip extending distally of the handle, an upper lumen and a lower lumen extending along the handle. The suction tip terminates near a distal end in a fluid confluence and at least one suction port. The clearing apparatus has a flexible rod body with a finger tab configured to tactilely slide the rod body to and fro in the upper lumen to move a distal tip of the rod body through the confluence and the at least one suction port. A method is also provided.

### RELATED PATENT DATA

This utility patent application claims priority to U.S. Provisional Patent Application Ser. No. 63/527,725, filed Jul. 19, 2023, entitled “Surgical Suction Tube and Method”, the disclosures of which are incorporated herein by reference.

### TECHNICAL FIELD

This disclosure relates to the field of surgical instruments used for removing debris from within a surgical operative field. More particularly, this disclosure pertains to tubular suction devices for medical, surgical, veterinary, and dental use having a mechanism for clearing debris from an intake of the device.

### BACKGROUND OF THE INVENTION

Techniques are known for removing debris from an inlet of a suction device being used in a surgical operative environment. However, such devices are often encumbered by tethers to suction devices and lighting power supplies and are not self-contained. Accordingly, improvements are needed to improve ergonomics, increase lighted visibility, and realize single-use devices that can be disposable or self-contained.

### SUMMARY OF THE INVENTION

An apparatus and method are provided to enable a surgeon to self-remove obstructions from a distal end of a surgical suction device. Furthermore, an apparatus and method are provided to enhance visibility while removing obstructions and debris from a surgical site.

According to one aspect, surgical suction device is provided having an elongate unitary construction lumen body and a clearing apparatus. The lumen body has a handle and a suction tip extending distally of the handle, an upper lumen and a lower lumen extending along the handle. The suction tip terminates near a distal end in a fluid confluence and at least one suction port. The clearing apparatus has a flexible rod body with a finger tab configured to tactilely slide the rod body to and fro in the upper lumen to move a distal tip of the rod body through the confluence and the at least one suction port.

According to another aspect, a surgical suction device is provided having an elongate lumen body and a clearing apparatus. The elongate lumen body has a handle and a suction tip extending distally of the handle, an upper lumen and a lower lumen extending along the handle in fluid confluence with one another, and further in fluid communication with at least one suction port. The clearing apparatus has a flexible rod body with a finger tab configured to tactilely slide the rod body to and fro in the upper lumen to move a distal tip of the rod body through the confluence and the at least one suction port.

According to yet another aspect, a method is provided for removing debris from a surgical site. The method includes providing a surgical suction device having an elongate lumen body having a handle and a suction tip extending distally of the handle, an upper lumen and a lower lumen extending along the handle in fluid confluence with one another, and further in fluid communication with at least one suction port; and a clearing apparatus having a flexible rod body with a finger tab configured to tactilely slide the rod body to and fro in the upper lumen to move a distal tip of the rod body through the confluence and the at least one suction port; inserting the distal end of the suction tip into a surgical environment; applying suction via the lower lumen to the suction tip to suction fluid from a surgical site in the surgical environment; and concurrent with applying the suction, tactilely sliding the rod body to and fro in the upper lumen to move a distal tip of the rod body through the confluence and the at least one suction port to clear debris from the at least one suction port of the suction tip and the lower lumen.

### BRIEF DESCRIPTION OF THE DRAWINGS

FIG. 1 is a perspective view from a leading end and above of a surgical suction device having a unitary construction with a manual clearing tip according to one version.

FIG. 2 is a side view of the surgical suction device of FIG. 1.

FIG. 3 is a centerline vertical sectional view of the surgical suction device of FIG. 2 taken along line 3-3 of FIG. 4.

FIG. 4 is a vertical sectional view taken along 4-4 of FIG. 3.

FIG. 5 is a vertical sectional view taken along line 5-5 of FIG. 3.

FIG. 6 is a perspective view from a leading end and above of another surgical suction device having a unitary construction with a lighted manual clearing tip according to another version.

FIG. 7 is a side view of the surgical suction device of FIG. 6.

FIG. 8 is a centerline vertical sectional view of the surgical suction device of FIG. 7 taken along line 8-8 of FIG. 9.

FIG. 9 is a vertical sectional view taken along 9-9 of FIG. 8.

FIG. 10 is an enlarged vertical sectional view taken from encircled region 10 of FIG. 8.

FIG. 11 is a vertical sectional view taken along line 11-11 of FIG. 10.

FIG. 12 is a vertical sectional view taken along line 12-12 of FIG. 10.

FIG. 13 is a side view of even another surgical suction device having a unitary construction and a lighted manual clearing tip with one alternative power supply.

FIG. 14 is a vertical sectional view taken along line 14-14 of FIG. 13.

FIG. 15 is a perspective view from a leading end and above of a surgical suction device having an assembled component construction with a manual clearing tip according to yet another version.

FIG. 16 is vertical partial centerline sectional view taken along line 16-16 of FIG. 17.

FIG. 17 is a vertical partial centerline view taken along line 17-17 of FIG. 16.

### DETAILED DESCRIPTION OF THE INVENTION

This disclosure of the invention is submitted in furtherance of the constitutional purposes of the U.S. Patent Laws “to promote the progress of science and useful arts” (Article 1, Section 8).

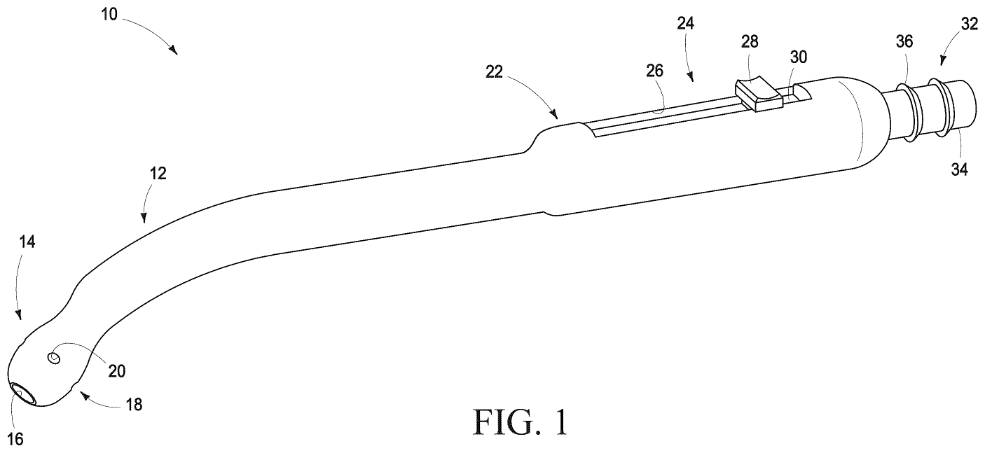

FIG. 1 is a perspective view from a leading end and above of a surgical suction device 10 having a unitary construction with a manual clearing tip according to one version. More particularly, surgical suction apparatus 10 includes a distal suction tip 12 terminating in a suction head 14, a handle, or tactile body 22, and a vacuum source attachment end fitting 32. A clearing tip rod 30 extends coaxially within handle 22 and suction tip 12 for reciprocation responsive to a user tacitly manipulating rod 30 by sliding finger tab 28 to and fro within slot 26 so as to provide a clearing apparatus 24 for main suction bore 16 and array 18 of individual peripheral suction bores 20 on suction head 18. In operation, surgical suction apparatus is affixed onto an end of a flexible hose suction source (not shown) by inserting vacuum source end fitting 32 with such tube in a force fit and sealing relationship.

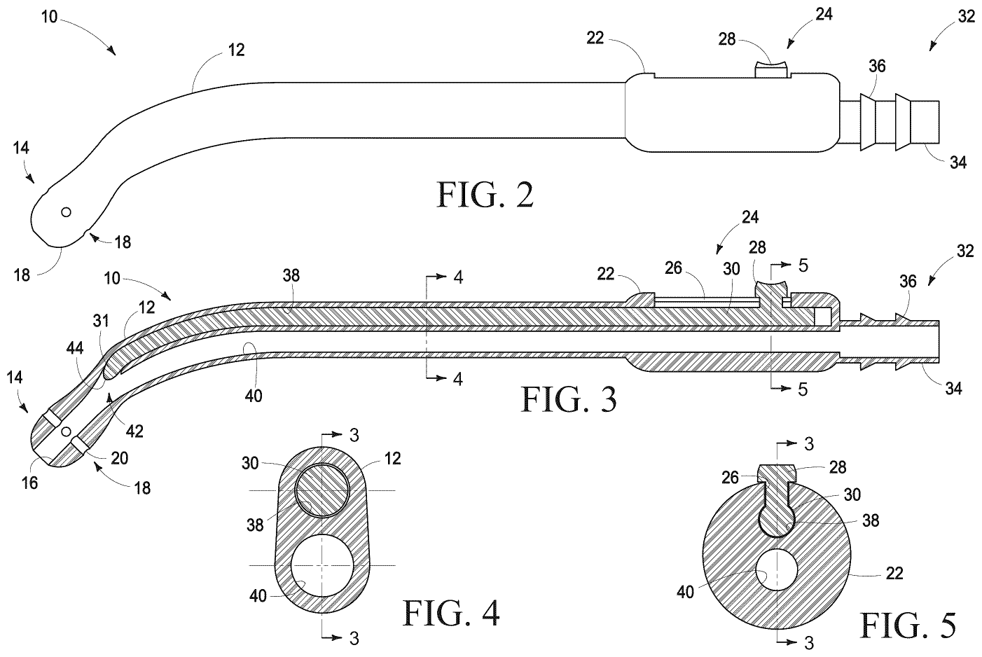

FIG. 2 is a side view of the surgical suction device 10 of FIG. 1 illustrating a contiguous transition between suction tip 12 and handle 22. Barbed end fitting 34 is formed contiguously on a proximal end of handle 22 and suction head 14 is provided integrally on a distal end of suction tip 12. Finger slide, or tab 28 is shown extending from handle 22 operative by a using with tactile movements fore and aft to unclog suction head 14 during operation.

FIG. 3 is a centerline vertical sectional view of the surgical suction device 10 of FIG. 2 taken along line 3-3 of FIG. 4. Suction source fitting 32 comprises a tubular cylindrical end fitting 34 having a pair of racially outwardly extending circumferential barbs 36 that are received in assembly within an inner bore of a flexible plastic vacuum source supply tube (not shown). The pair of circumferential barbed ridges, or circumferential barbs 36 extend radially outwardly from cylindrical main body of tube end fitting 34 and are sized to be received within an inner bore of a flexible vacuum source line in a slightly interference fit that secures fitting 32 within such tube when received therein.

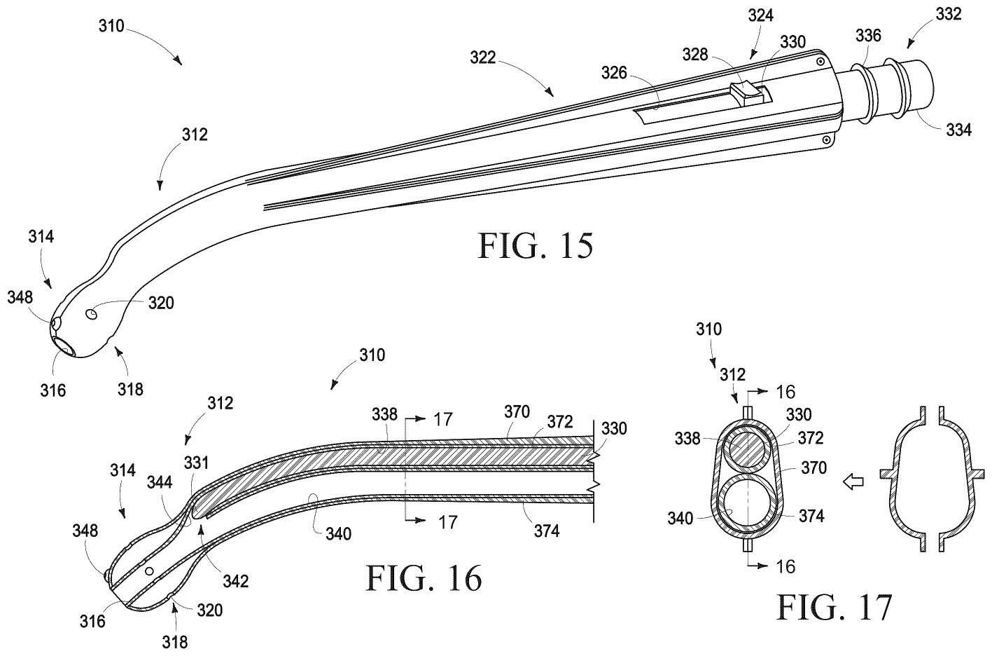

As shown in FIG. 3, surgical suction device 10 has an upper lumen 38 and a lower lumen 40 extending from handle 22 to suction tip 12. Upper lumen 38 receives a rod body 30 having a finger manipulated slide, or tab 28 along slot 26 configured to enable a user to extend a rod tip 31 from lumen 38 into a confluence 42 via aperture 44 into lumen 40 and out through bore 16 and past bores 20 of array 18 in order to clear any debris present in bores 16 and 20 of head 14. A unitary construction is depicted for surgical suction device 10 with handle 22, suction tip 12, head 14 and vacuum source end fitting 32 being formed from a single piece of molded plastic forming bores, or lumens 38 and 40 internally therein. Optionally, surgical suction device 10 can be constructed from a plurality of components that are affixed together, such as shown in the depicted in FIGS. 15-17. One suitable plastic for manufacturing device 10 is ISO 10993 biocompatible materials such as certified resin materials. Exemplary materials can include polyamide, polyurethane, polycarbonate, and fluoropolymers (such as polytetrafluoroethylene (PTFE)). Other suitable materials that can be sterilized are also desirable capable of being sterilized using ethylene oxide, gamma radiation, and/or steam. Further optionally, any suitable structural material such as any plastic, composite, metal, or metal alloy can be used.

FIG. 4 is a vertical sectional view taken along 4-4 of FIG. 3 showing suction tip 12 having rod body 30 coaxially assembled within upper lumen, or bore 38 in a sliding fit. Lower lumen 40 is shown beneath upper lumen and is sized to apply a vacuum at head 14 (see FIG. 3) to draw debris and fluids from a surgical site out via end fitting 32 to an external vacuum source and container (not shown).

FIG. 5 is a vertical sectional view taken along line 5-5 of FIG. 3 showing tab 28 received for axial sliding movement within slot 26 and integrally formed with rod 30 configured for sliding movement within bore 38. Lower lumen, or bore 40 is shown below upper lumen, or bore 38 within handle 22.

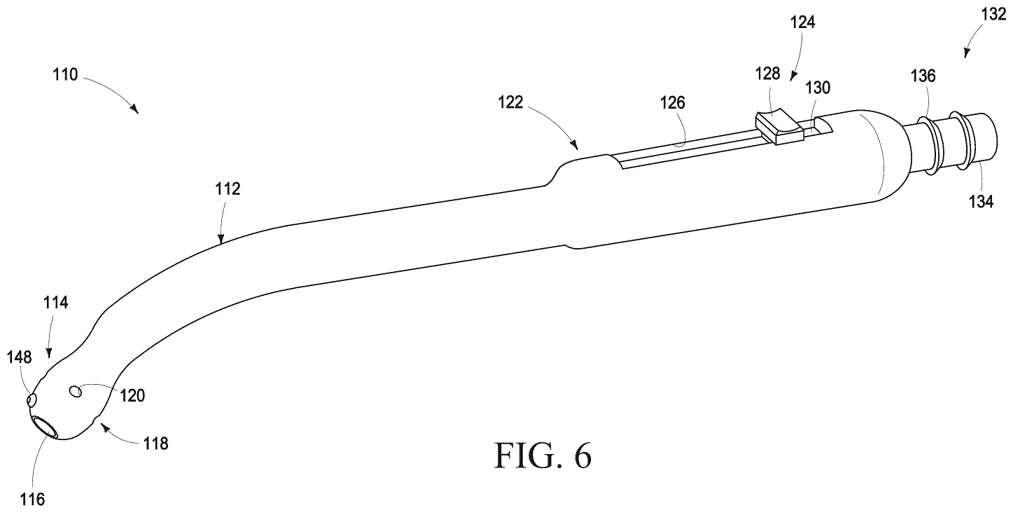

FIG. 6 is a perspective view from a leading end and above of another surgical suction device 110 having a unitary construction with a lighted manual clearing suction tip 114 according to another version. More particularly, a micro Light Emitting Diode (LED) light source 148 is provided on a forward top portion of suction head 114 configured to visually aid a user when manipulating head 114 and suction bores 116 and 120 (in array 118). Head 114 is formed in a distal end of suction tip 112 while handle 122 is formed at a proximal end of tip 112. End fitting 132 is affixed to a proximal end of handle 122 presenting barbs 136 on tubular end 134 for affixation onto a flexible vacuum suction supply line source. Clearing apparatus 124 is presented for use by a user holding handle 122 during use of suction apparatus 110 and finger tab 128 has a concave top surface that mates with a user's digit, such as a thumb tip enabling a user to slide tab 128 and rod 130 in axial reciprocation within slot 126.

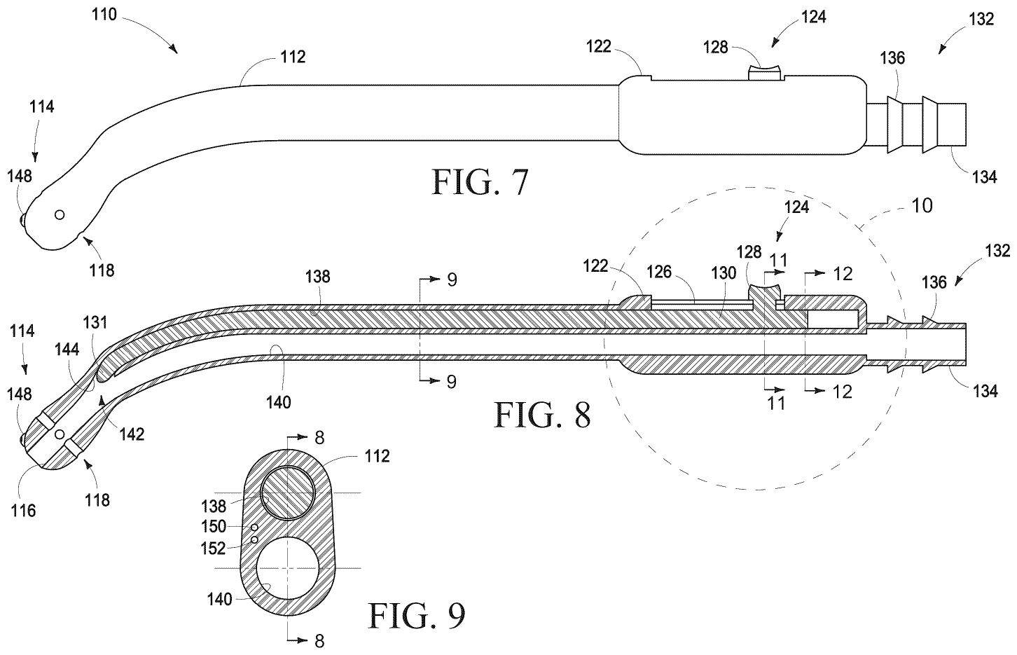

FIG. 7 is a side view of the surgical suction device 110 of FIG. 6 illustrating in side elevational view the orientation of micro LED light source 148 along a leading end of suction head 114 at a distal end of suction tip 112 forward of array 118. A user grasping handle 122 engages finger tab 128 of clearing apparatus 124 while end fitting 32 is affixed with barbs 136 on tubular fitting 134 inside of a flexible suction supply line (not shown).

FIG. 8 is a centerline vertical sectional view of the surgical suction device 110 of FIG. 7 taken along line 8-8 of FIG. 9. Suction source fitting 132 comprises a tubular cylindrical end fitting 134 having a pair of racially outwardly extending circumferential barbs 136 that are received in assembly within an inner bore of a flexible plastic vacuum source supply tube (not shown). The pair of circumferential barbed ridges, or circumferential barbs 136 extend radially outwardly from cylindrical main body of tube end fitting 134 and are sized to be received within an inner bore of a flexible vacuum source line in a slightly interference fit that secures fitting 132 within such tube when received therein. A micro LED light source 148 is shown affixed to a distal end of suction head 114 to aid in a user visualizing a suction sight.

As shown in FIG. 8, surgical suction device 110 has an upper lumen 138 and a lower lumen 140 extending from handle 122 to suction tip 112. Upper lumen 138 receives a rod body 130 having a finger manipulated slide, or tab 128 along slot 126 configured to enable a user to extend a rod tip 131 from lumen 138 into a confluence 142 via aperture 144 into lumen 140 and out through bore 116 and past bores 120 (see FIG. 6) of array 118 in order to clear any debris present in bores 116 and 120 of head 114. Bores 116 and 120 each form one of a plurality of suction ports in fluid communication with suction applied via lower lumen 140 with a suction source (not shown). A unitary construction is depicted for surgical suction device 110 with handle 122, suction tip 112, head 114 and vacuum source end fitting 132 being formed from a single piece of molded plastic forming bores, or lumens 138 and 140 internally therein. Optionally, surgical suction device 110 can be constructed from a plurality of components that are affixed together, such as shown in the depicted in FIGS. 15-17. One suitable plastic is ISO 10993 biocompatible certified resin material such as a polyamide or a polyurethane. Any other previously mentioned alternative material mentioned with reference to FIG. 3 can also be utilized.

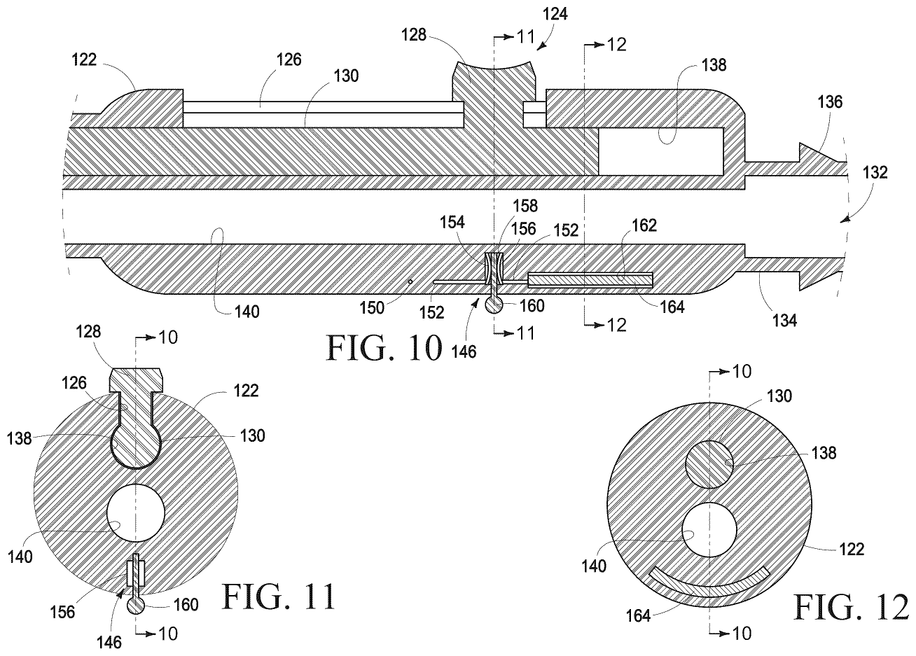

FIG. 9 is a vertical sectional view taken along 9-9 of FIG. 8 showing suction tip 112 having rod body 130 coaxially assembled within upper lumen, or bore 138 in a sliding fit. Lower lumen 140 is shown beneath upper lumen and is sized to apply a vacuum at head 114 (see FIG. 7) to draw debris and fluids from a surgical site out via end fitting 132 to an external vacuum source and container (not shown). Individual insulated conductive wires 150 and 152 are in-molded within suction tip 112 and extend from micro-LED light source 148 and Optionally 164 (see FIGS. 10 and 12) to form a closed-circuit electrical circuit between such battery and light source.

FIG. 10 is an enlarged vertical sectional view taken from encircled region 10 of FIG. 8 illustrating in partial enlarged detail components within handle 22. More particularly, rod 30 is shown sized to be slidably received within bore, or lumen 138 and finger tab 128 sized to be received in complementary relation to slide within slot 126 during a cleaning operation. End fitting 132 is provided in fluid communication with lumen 140 with barb 138 on tubular fitting 134 configured to secure to a flexible vacuum line.

As shown in FIG. 10, a power supply is electrically coupled via positive and negative terminals and insulated conductive wires, such as wires 150 and 152 (see FIG. 9) using a Lithium Ion (Li-ion) battery 162 supported within a semi-circumferential cavity 162 formed within handle 122. Optionally, a thin film 3 Volt zinc/magnesium battery can be used. A single use electrical switch 146 is provided within a rectangular cavity 158 formed in handle 12 configured to receive a pair of rectangular, arcuate conductive metal spring contacts 154 and 156 that engage together in electrically conductive relation after an insulative first-use strip, or pull tab 160 is removed from therebetween. Disrupted ends of wire 152 are soldered to each contact 154 and 156 and when contacts 154 and 156 engage together (after pulling out pull tab 160 to form a completed electrical circuit between a battery and a light source. A removable cover, not shown, can be integrated into handle 122 with a snap fit to gain access to cavities 158 and 162 and to secure components of switch 146, battery 162 and wires 150 and 152 (see FIG. 9). One of a positive and a negative circuit leg is provided by wire 152 extending between battery 162 and micro-LED light source 148 (see FIG. 8). The remaining circuit leg 150 is not shown in this cross section and is provided proximate and alongside of wire 152. Such wires 150 and 152 move from a bottom position of handle 122 to a middle position as shown in FIG. 9 along a distal portion of handle 122. Wire 150 is shown in angled sectional view where the transition across the bottom of handle 122 occurs.

FIG. 11 is a vertical sectional view taken along line 11-11 of FIG. 10 showing tab 128 received for axial sliding movement within slot 126 and integrally formed with rod 130 and configured for sliding movement within upper lumen, or bore 138 of handle 122. Lower lumen, or bore 140 is shown below upper lumen 138 within handle 122. Initial use electrical switch 146 shows intermediate insulative pull tab 160 provided in front of conductive metal spring contact 156.

FIG. 12 is a vertical sectional view taken along line 12-12 of FIG. 10 illustrating placement of arcuate semi-cylindrical Lithium-Ion battery 164 within housing 122 relative to upper lumen 138 and lower lumen 140. Rod body 30 is shown coaxially received within upper lumen 138 for reciprocation.

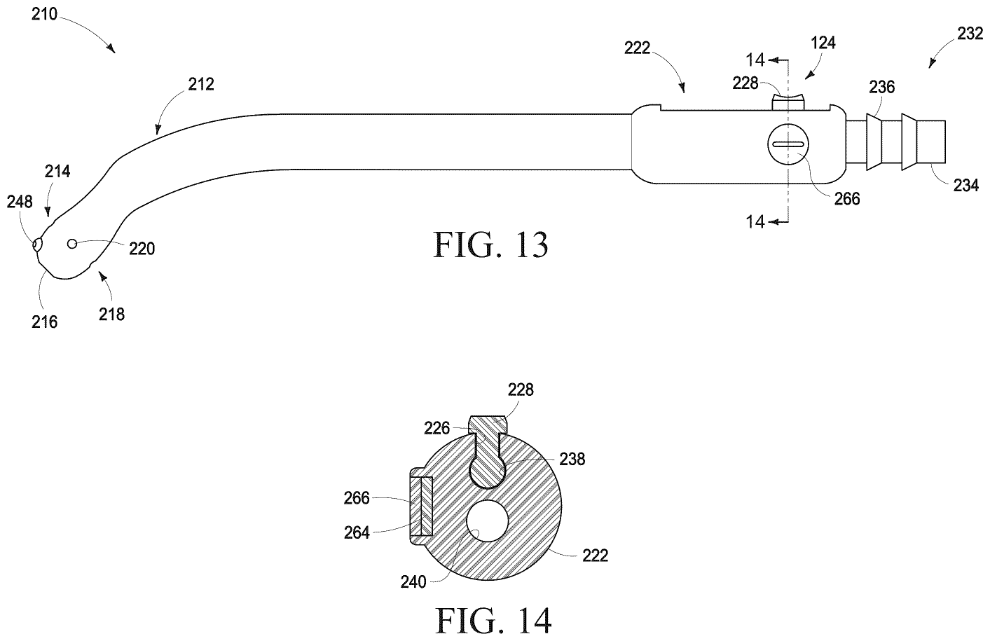

FIG. 13 is a side view of even another surgical suction device 210 having a unitary construction and a lighted manual clearing tip 214 with one alternative power supply. A micro LED light source 248 is provided at a leading end of suction head 214 at a distal end of suction tip 212 forward of radial array 218 of bores 220 and end suction bore 216. A user grasping handle 222 engages finger tab 228 of clearing apparatus 224 while end fitting 232 is affixed with barbs 236 on tubular fitting 234 inside of a flexible suction supply line (not shown). A button type battery cover 266 is provided in handle for accessing a power supply. Optionally, such button type battery can be molded inside of handle 222 for single use applications.

FIG. 14 is a vertical sectional view taken along line 14-14 of FIG. 13. Finger tab 228 is shown received for axial sliding movement within slot 226 and integrally formed with rod 230 and configured for sliding movement within upper lumen, or bore 238 of handle 222. Lower lumen, or bore 240 is shown below upper lumen 238 within handle 222. A button type battery 264 is retained within a recess of handle 222 using a plastic twist-lock cover 266.

FIG. 15 is a perspective view from a leading end and above of a surgical suction device 310 having an assembled multiple component construction with a manual clearing tip, or rod 330 according to yet another version. More particularly, surgical suction apparatus 310 includes a distal suction tip 312 terminating in a suction head 314, a handle, or tactile body 322, and a vacuum source attachment end fitting 332. Clearing tip rod 330 extends coaxially within handle 322 and suction tip 312 for reciprocation responsive to a user tacitly manipulating rod 330 by sliding finger tab 328 to and fro within slot 326 so as to provide a clearing apparatus 324 for main suction bore 316 and array 318 of individual peripheral suction bores 320 on suction head 318. In operation, surgical suction apparatus is affixed onto an end of a flexible hose suction source (not shown) by inserting vacuum source end fitting 332 having circumferential barbs 336 on tubular fitting 334 with such tube in a force fit and sealing relationship. A micro-LED light source 348 is also provided at a distal end of head 314.

FIG. 16 is vertical partial centerline sectional view taken along line 16-16 of FIG. 17 illustrating in partial vertical centerline sectional view. More particularly, suction head 314 on tip 312 of surgical suction device 310 has an upper lumen 338 and a lower lumen 340 along suction tip 312. Upper lumen 338 is formed in a separate upper tube 372, while lower lumen 340 is formed in another separate tube 374. Tubes 372 and 374 are housed within a two-piece outer housing 370 that is secured together using a plurality of fasteners. Upper lumen 338 of tube 372 receives a rod body 330 having a finger manipulated slide, or tab 328 (see FIG. 15) along slot 326 configured to enable a user to extend a rod tip 331 from lumen 338 into a confluence 342 via aperture 344 into lumen 340 and out through bore 316 and past bores 320 of array 318 in order to clear any debris present in bores 316 and 320 of head 314. A multiple component assembled construction is depicted for surgical suction device 310 with handle 322, suction tip 312, head 314 and vacuum source end fitting 332 being formed from a several pieces of secured together molded plastic that form bores, or lumens 338 and 340 internally therein. Optionally, surgical suction device 310 can be constructed from a unitary construction, such as shown in FIGS. 6-12. One suitable plastic is ISO 10993 biocompatible certified resin material such as a polyamide or a polyurethane. Any other previously mentioned alternative material mentioned with reference to FIG. 3 can also be utilized.

FIG. 17 is a vertical partial centerline view taken along line 17-17 of FIG. 16 showing in vertical sectional view the multiple component assembly of apparatus 310 along tip 312 showing lumen tubes 372 and 374 contained within two components outer shell 370. Bores 338 and 340 of tubes 372 and 374 provide for sliding rod body 330 and vacuum/debris evacuation, respectively.

Although not shown to drawing scale due to the slight degree of taper, in one construction of the devices of FIGS. 1-17 it is understood that the inner lumen is tapered from a smallest diameter near the tip end, or entrance rearward to the handle end in order to reduce any tendency for such lumen to clog with debris. For example, lower lumen 140 of FIG. 8 has a smaller inner diameter at a terminal end of the suction tip, or head 114 and expands slightly in diameter progressing towards end fitting 132.

The taper of lower lumen 140 of FIG. 8 would entail gradual enlargement of the inner diameter of the lower, suction lumen 140 extending in a downstream direction from the suction tip 114 towards the end fitting 132. This would comprise a gradual concentric enlargement of the inner lumen 140 from 4.2 mm at the distal suction opening to 6.2 mm at the proximal aspect of the device adjacent a suction supply source, by way of one exemplary construction. Other constructions, geometries and tapers are also contemplated. The 6.2 mm inner diameter is the inner diameter of lower lumen 140 at a point where lumen 140 attaches to a negative pressure suction tubing with a negative pressure vacuum source relative to atmospheric pressure. Such taper imparts favorable reduction in particulate or debris clogging of lumen 140 during operation in a surgical environment.

Lower lumen 140 increases in diameter from a distal end of the tip head 114 to an exit, or proximal end near the end fitting 138. Preferably, an inner diameter of lumen 140 is tapered to increase in diameter by 1/100th of a centimeter for each axial centimeter of length along lower lumen 140. Other alternative constructions are also possible including varying inner diameter tapers or changing degrees of tapered for lumen 140 as needed to mitigate clogging of debris within lumen 140 during use. Optionally, inner lumen 140 can have a constant inner diameter.

In compliance with the statute, the subject matter disclosed herein has been described in language more or less specific as to structural and methodical features. It is to be understood, however, that the claims are not limited to the specific features shown and described, since the means herein disclosed comprise example embodiments. The claims are thus to be afforded full scope as literally worded, and to be appropriately interpreted in accordance with the doctrine of equivalents.

The terms “a”, “an”, and “the” as used in the claims herein are used in conformance with long-standing claim drafting practice and not in a limiting way. Unless specifically set forth herein, the terms “a”, “an”, and “the” are not limited to one of such elements, but instead mean “at least one”.

I/We claim:

1. A surgical suction device, comprising:
an elongate unitary construction lumen body having a handle and a suction tip extending distally of the handle, an upper lumen and a lower lumen extending along the handle and the suction tip terminating near a distal end in a fluid confluence and at least one suction port; and
a clearing apparatus having a flexible rod body with a finger tab configured to tactilely slide the rod body to and fro in the upper lumen to move a distal tip of the rod body through the confluence and the at least one suction port.

2. The surgical suction device of claim 1, further comprising a light source provide proximate a distal end of the suction tip.

3. The surgical suction device of claim 2, further comprising a battery carried in the handle and electrically coupled with the light source.

4. The surgical suction device of claim 3, further comprising a switch affixed in assembly to interrupt power supply to the light source and having a pull tab configured to empower the light source with the battery upon removal of the pull tab.

5. The surgical suction device of claim 3, wherein the lower lumen comprises a decreasing inner diameter taper provided intermediate and extending in a direction from the distal end of the suction tip to the handle.

6. The surgical suction device of claim 5, wherein the inner diameter taper of the lower lumen decreases in diameter from the tip end towards the handle by 1/100th of a centimeter for each axial centimeter of length.

7. The surgical suction device of claim 1, wherein the at least one suction port of the suction tip comprises a distal end bore of the lower lumen.

8. The surgical suction device of claim 1, wherein the at least one suction port of the suction tip comprises a bore extending radially outwardly from the lower lumen through the suction tip.

9. The surgical suction device of claim 8, further comprising an array of radially outwardly extending bores extending from the lower lumen through the suction tip.

10. The surgical suction device of claim 8, further comprising a distal end of the lower lumen further providing another port at a distal end of the suction tip.

11. A surgical suction device, comprising:
an elongate lumen body having a handle and a suction tip extending distally of the handle, an upper lumen and a lower lumen extending along the handle in fluid confluence with one another, and further in fluid communication with at least one suction port; and
a clearing apparatus having a flexible rod body with a finger tab configured to tactilely slide the rod body to and fro in the upper lumen to move a distal tip of the rod body through the confluence and the at least one suction port.

12. The surgical suction device of claim 11, wherein the at least one suction port comprises a distal end bore of the lower lumen provided at a distal end of the suction tip.

13. The surgical suction device of claim 12, wherein the at least one suction port further comprises a bore extending radially outwardly from the lower lumen through the suction tip.

14. The surgical suction device of claim 13, further comprising an array of radially outwardly extending bores extending from the lower lumen through the suction tip.

15. The surgical suction device of claim 11, wherein the lower lumen comprises a decreasing inner diameter taper provided intermediate and extending in a direction from the distal end of the suction tip toward the handle.

16. The surgical suction device of claim 15, further comprising a light source provide proximate the distal end of the suction tip and superior to the distal end of the suction tip.

17. A method of suctioning debris during a surgical procedure, comprising:
providing a surgical suction device having an elongate lumen body having a handle and a suction tip extending distally of the handle, an upper lumen and a lower lumen extending along the handle in fluid confluence with one another, and further in fluid communication with at least one suction port; and a clearing apparatus having a flexible rod body with a finger tab configured to tactilely slide the rod body to and fro in the upper lumen to move a distal tip of the rod body through the confluence and the at least one suction port;
inserting the distal end of the suction tip into a surgical environment;
applying suction via the lower lumen to the suction tip to suction fluid from a surgical site in the surgical environment; and
concurrent with applying the suction, tactilely sliding the rod body to and fro in the upper lumen to move a distal tip of the rod body through the confluence and the at least one suction port to clear debris from the at least one suction port of the suction tip and the lower lumen.

18. The method of claim 17, wherein the surgical suction device further comprises a light source provided proximate and superior to the distal end of the suction tip, and further comprising illuminating the surgical site with the light source.

19. The method of claim 17, wherein the rod body is a flexible rod body, the upper lumen is an arcuate upper lumen, and tactilely sliding the rod body comprises guiding the flexible rod body along the arcuate upper lumen into the lower lumen in the confluence.

20. The method of claim 19, wherein the lower lumen is tapered in increasing diameter extending from the distal end of the suction tip to the handle and the at least one suction port comprises an array of suction ports about the suction tip.
